SEQUENCE LISTING (19 sequences)

SEQ ID NO: 1 - DNA, 20 bp, Artificial Sequence
  Synthetic construct of DNA1
cctaacttgacgatacagcg

SEQ ID NO: 2 - DNA, 20 bp, Artificial Sequence
  Synthetic construct of DNA2
tggaatgctggtggcgtagg

SEQ ID NO: 3 - DNA, 18 bp, Artificial Sequence
  Synthetic construct of DNA1′
cgctgtatcgtcaagtta

SEQ ID NO: 4 - DNA, 18 bp, Artificial Sequence
  Synthetic construct of DNA2′
cctacgccaccagcattc

SEQ ID NO: 5 - DNA, 18 bp, Artificial Sequence
  Synthetic construct of DNA1
taacttgacgatacagcg

SEQ ID NO: 6 - DNA, 18 bp, Artificial Sequence
  Synthetic construct of DNA2
gaatgctggtggcgtagg

SEQ ID NO: 7 - DNA, 15 bp, Artificial Sequence
  Synthetic construct of Aptamer1
ggttggtgtggttgg

SEQ ID NO: 8 - DNA, 28 bp, Artificial Sequence
  Synthetic construct of Aptamer2
agtccgtggtagggcaggttggggtgac

SEQ ID NO: 9 - DNA, 39 bp, Artificial Sequence
  Synthetic construct of Aptamer1-DNA1
ggttggtgtggttggtttttttaacttgacgatacagcg

SEQ ID NO: 10 - DNA, 52 bp, Artificial Sequence
  Synthetic construct of Aptamer2-DNA2
agtccgtggtagggcaggttggggtgacttttttgaatgctggtggcgtagg

SEQ ID NO: 11 - protein, 288 aa, Artificial Sequence
  Synthetic construct of TEVP-SpyTag
MGSSHHHHHHGSWSHPQFEKKLGGGSGGGSAHIVMVDAYKPTKGGGSGGGSEFESLFKGPRDYNPISSTICHLTNESDGHTTSLYGIGFGPFIITNKHLFRRNNGTLVVQSLHGVFKVKNTTTLQQHLIDGRDMIIIRMPKDFPPFPQKLKFREPQREERICLVTTNFQTKSMSSMVSDTSCTFPSGDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 12 - protein, 142 aa, Artificial Sequence
  Synthetic construct (SpyCatcher-AzF)
  MISC_FEATURE (17)..(17): Xaa is any amino acid
MGSSHHHHHHGSGGGSXKLGGGSGGGSAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 13 - protein, 498 aa, Artificial Sequence
  Synthetic construct (BLZ-AzF)
  MISC_FEATURE (25)..(25): Xaa is any amino acid
MKYLLPTAAAGLLLLAAQPAMAMGXGGSGGSAGVMTGAKFTQIQFGMTRQQVLDIAGAENCETGGSFGDSIHCRGHAAGDYYAYATFGFTSAAADAKVDSKSQEKLLAPSAPTLTLAKFNQVTVGMTRAQVLATVGQGSCTTWSEYYPAYPSTAGVTLSLSCFDVDGYSSTGAYRGSAHLWFTDGVLQGKRQWDLVGSGGGSGGGSENLYFQGGGGSGGGSKLMDERNRQIAEIGASLIKHWGGGGGHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTTPVAMATTLRKLLTGELLTLASRQQLIDWMEADKVAGPLLRSALPAGWFIADKSGAGERGSRGIIAALGPDGEPSRIVVIYTTGSQALEHHHHHH

SEQ ID NO: 14 - protein, 469 aa, Artificial Sequence
  Synthetic construct (Trastuzumab, heavy chain)
MGWSCIILFLVATATGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 233 aa, Artificial Sequence
  Synthetic construct (Trastuzumab, light chain)
MGWSCIILFLVATATGVHSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 467 aa, Artificial Sequence
  Synthetic construct (Pertuzumab, heavy chain)
MGWSCIILFLVATATGVHSEVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 233 aa, Artificial Sequence
  Synthetic construct (Pertuzumab, light chain)
MGWSCIILFLVATATGVHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 122 aa, Artificial Sequence
  Synthetic construct (hCG, alpha-subunit)
MDYYRKYAAIFLVTLSVFLHVLHSAPDVQDCPECTLQENPLFSQPGAPILQCMGCCFSRAYPTPLRSKKTMLVQKNVTSESTCCVAKSYNRVTVMGGFKVENHTACHCSTCYYHKSHHHHHH

SEQ ID NO: 19 - protein, 173 aa, Artificial Sequence
  Synthetic construct (hCG, beta-subunit)
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQGSHHHHHH

(19) United States
(12) Patent Application Publication
(10) Pub. No.: US 2024/0151714 A1
(43) Pub. Date: May 9, 2024

(54) TARGET ANALYTE DETECTION METHOD BASED ON PROXIMITY PROTEOLYSIS REACTION

(71) Applicant: AJOU UNIVERSITY INDUSTRY-ACADEMIC COOPERATION FOUNDATION, Suwon-si, Gyeonggi-do (KR)

(72) Inventors: Tae Hyeon YOO, Yongin-si, Gyeonggi-do (KR); Hyeon Ji PARK, Suwon-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 18/281,460

(22) PCT Filed: Mar. 14, 2022
(86) PCT No.: PCT/KR2022/003525

§ 371 Date: Sep. 11, 2023

(30) Foreign Application Priority Data
Mar. 12, 2021 (KR) 10-2021-0032756

(51) Int. Cl. G01N 33/53; G01N 33/573; G01N 33/68; G01N 33/74; G01N 33/76

(52) U.S. Cl. CPC G01N 33/5308; G01N 33/573; G01N 33/6887; G01N 33/74; G01N 33/76; G01N 2333/4712; G01N 2333/59; G01N 2333/71; G01N 2333/96463

(57) ABSTRACT

The present invention relates to a target analyte detection composition based on a proximity proteolysis reaction and a target analyte detection method using same. More specifically, the present invention relates to a method for detecting a target analyte, the method comprising a step in which when a first binder and a second binder bind to the target analyte, hybridization is made between ssDNA linked to the first binder and ssDNA linked to a protease and between ssDNA linked to the second binder and ssDNA linked to a zymogen, whereby a signal generated by a proximity proteolysis reaction between the protease and the zymogen is detected.

### TECHNICAL FIELD

The present invention relates to a composition for detecting a target substance based on proximity proteolysis reaction and a method for detecting a target substance using the same, and more specifically, to a method for detecting a target substance including binding a first binder and a second binder to a target substance, hybridizing ssDNA linked to the first binder with ssDNA linked to a protease, hybridizing ssDNA linked to the second binder with ssDNA linked to an enzyme source (zymogen), and detecting a signal generated by a proximity proteolysis reaction between the protease and the enzyme source.

### BACKGROUND ART

Various biological molecules, including nucleic acids, proteins, and small molecules, show the physiological state of organisms and act as biomarkers of diseases. Assays for detecting or quantifying biological molecules are essential to the clinical field and various assays have been developed (Wu, L. & Qu, X. Chem Soc Rev 44, 2963-2997, doi:10.1039/c4cs00370e (2015); Sanavio, B. & Krol, S. Front Bioeng Biotechnol 3, 20, doi:10.3389/fbio e.2015.00020 (2015)). Simple homogeneous assays for nucleic acids using specific hybridization of DNA strands based on the Watson-Crick base pairing principle have been developed, contributing to the early detection of pathogens and abnormal cells. On the other hand, heterogenous assays involving solid surfaces, such as enzyme-linked immunosorbent assay (ELISA) and modified versions thereof, have been standards for detecting proteins and small molecules for decades (Zhang, S., et al., Analyst 139, 439-445, doi:10.1039/c3an01835k (2014)). Although these methods meet the essential features of diagnostic tools, such as robustness, sensitivity, and specificity, the typical procedure for these assays includes a plurality of steps for target binding and elimination of non-specific interactions, typically requires trained personnel or automated instruments, and takes more than one day. Therefore, heterogeneous assays are not suitable as point-of-care methods. Recently, efforts have been made to research and develop point-of-care tests that contribute to early diagnosis of diseases in resource-limited environments.

Typically, a method optimized for point-of-care diagnosis is an analysis performed in a homogeneous phase. Various strategies have been suggested for designing homologous assays to detect proteins and small molecules, including inducing molecular assembly in the presence of a target molecule (Liu, H. et al. Theranostics 6, 54-64, doi:10.7150/thno.13159 (2016); Hwang, B. B., et al., Commun Biol 3, 8, doi:10.1038/s42003-019-0723-9 (2020)). The colocalization of the sensor generates a detectable signal, allowing the assay to be performed in the liquid phase with minimal background signal. Forster resonance energy transfer pairs and split proteins were used to monitor molecular interactions. When these molecules are covalently or physically linked to two binders that target independent regions of the molecule, the resulting molecules can detect the target in a homogeneous phase. The reaction rate can be improved by locating the reactants close to each other to increase effective concentrations thereof. This principle of reaction facilitation based on proximity has been used to design chemical and biological reactions to analyze various molecules and molecular interactions such as proteins, antibodies, and nucleic acids.

In this regard, a nucleic acid analysis method with simplicity and high sensitivity based on a novel proximity-enhancing reaction called “proximity proteolysis reaction (PPR)” is known (Park, H. J. & Yoo, T. H. ACS Sens 3, 2066-2070, doi:10.1021/acssensors.8b00821 (2018)), but there has been no report on a method for detecting target substances such as proteins and small molecules using this method.

Accordingly, as a result of diligent efforts to detect target substances such as proteins and small molecules with high sensitivity even at low concentrations, the present inventors have found that a target substance may be easily and quickly detected even at a sub-nanomolar target substance concentration when the target substance is detected using a first binder and a second binder binding to the target substance, and the proximity proteolytic reaction between a protease and zymogen linked through hybridization between each binder and ssDNA, thereby completing the present invention.

The information disclosed in this Background section is provided only for enhancement of understanding of the background of the present invention, and therefore it may not include information that forms the prior art that is already obvious to those skilled in the art.

### DISCLOSURE

Therefore, it is an object of the present invention to provide a composition for detecting a target substance capable of rapidly detecting target materials such as proteins and small molecules with high sensitivity, and a method for detecting a target substance using the same.

In accordance with one aspect of the present invention, the above and other objects can be accomplished by the provision of a composition for detecting a target substance including i) a first DNA-first binder conjugate in which a first DNA is linked to a first binder, ii) a first DNA'-protease conjugate in which a first DNA′ having a sequence complementary to the first DNA is linked to a protease, iii) a second DNA-second binder conjugate in which a second DNA is linked to a second binder, iv) a second DNA′-zymogen conjugate in which a second DNA′ having a sequence complementary to the second DNA is linked to an enzyme source (zymogen), and v) a substrate specific to the enzyme source.

In accordance with another aspect of the present invention, provided is a method of detecting a target substance including (a) mixing the composition with a sample containing a target substance, (b) binding the target substance to the first binder of the first DNA-first binder conjugate, binding the target substance to the second binder of the second DNA-second binder conjugate, hybridizing the first DNA linked to the first binder with the first DNA′ linked to a protease, and hybridizing the second DNA linked to the second binder with the second DNA′ linked to an enzyme source (zymogen), and (c) detecting a signal generated by a proximity proteolysis reaction between the first DNA′-protease conjugate hybridized with the first DNA of the first binder and the second DNA′-zymogen conjugate hybridized with the second DNA of the second binder.

In accordance with another aspect of the present invention, provided is a method of detecting a small molecule including (a) mixing a composition further containing an anti-small molecule with a sample containing a small molecule, (b) hybridizing the first DNA linked to the first binder with the first DNA′ linked to a protease and hybridizing the second DNA linked to the second binder with the second DNA′ linked to an enzyme source (zymogen), and (c) detecting whether or not the small molecule is present depending on the presence or absence of a signal generated by a proximity proteolysis reaction between the hybridized first DNA′-protease conjugate and the hybridized second DNA′-zymogen conjugate.

### DESCRIPTION OF DRAWINGS

FIG. 1 is a schematic diagram illustrating a typical PPR-based homogeneous assay. FIG. 1A is a schematic diagram illustrating a PPR-based homogeneous assay for detecting various targets. Engineered β-lactamase zymogen (BLZ) can be activated by a tobacco etch virus protease (TEVP). TEVP was linked to the first binder (Binder1) through hybridization between the first DNA′ (DNA1′) and the first DNA (DNA1), and BLZ was linked to the second binder (Binder2) through hybridization between the second DNA′ (DNA2′) and the second DNA (DNA2). FIG. 1B shows a procedure for a typical PPR-based homogeneous assay. Four conjugates (TEVP-first DNA′, BLZ-second DNA′, first binder-first DNA and second binder-second DNA) and a chromogenic substrate for β-lactamase (CENTA™) were mixed with the samples in a one-step process and the change in absorbance at 405 nm was measured at 37° C. after 1 hour.

FIG. 2 shows detection of the ectodomain of HER2 in solution. FIG. 2A is a schematic diagram illustrating a PPR-based homologous assay to detect the ectodomain of HER2. Trastuzumab-first DNA and pertuzumab-second DNA conjugates were used as binders for the ectodomain of HER2. FIG. 2B shows the linking of proteolytic pairs of TEVP and BLZ to trastuzumab and pertuzumab, respectively, using hybridization between complementary ssDNAs. Only samples containing both trastuzumab-first DNA and pertuzumab-second DNA exhibited a significant increase in absorbance signals in TEV-first DNA′ and BLZ-second DNA′ (T: trastuzumab, P: pertuzumab, T-D1: trastuzumab-first DNA, P-D2: pertuzumab-second DNA). FIG. 2C shows a curve of absorbance signal at 405 nm as a function of time depending on ectodomain concentration of HER2 using four ssDNA conjugates (TEVP-first DNA′: 20 nM, BLZ-second DNA′: 20 nM), trastuzumab-first DNA: 5 nM, pertuzumab-second DNA: 5 nM) and 400 pM CENTA™ in a reaction buffer (137 mM NaCl, 2.7 mM KCl, 10 mM Na₂HPO₄, 2 mM KH₂PO₄, 10 mM DTT, 40 mM MgCl₂, 0.5% (w/v) BSA; pH 7.4). FIG. 2D shows the relationship between the absorbance signal and the HER2 concentration which results from the assay of HER2 detection. The inset graph shows a linear relationship in the range of 0 to 1.25 nM of the ectodomain of HER2.

FIG. 3 shows detection of HER2 in cell membranes. FIG. 3A is a schematic diagram illustrating HER2 detection in cell membranes. Four conjugates (TEVP-first DNA′, BLZ-second DNA′, trastuzumab-first DNA, pertuzumab-second DNA) and CENTA™ were added to the solution containing suspended cells, and the absorbance signal was measured 1 hour later. FIG. 3B shows the signal for HER2-expressing cells measured by a PPR-based homologous assay for HER2. FIG. 3C shows analysis of HER2-expressing cells using flow cytometry. Cells were incubated along with trastuzumab and then labeled with goat anti-human IgG conjugated with a fluorescent dye (Alex Fluor 488). FIG. 3D shows the relationship between the absorbance signal obtained using a PPR-based homologous assay for HER2 and the number of BT-474 cells.

FIG. 4 shows the detection of cTnI. FIG. 4A is a schematic diagram illustrating cTnI detection. FIG. 4B shows the relationship between absorbance signal and cTnI concentration. The inset graph shows the linear relationship for cTnI in the range of 0-5.0 nM. The assay was performed using the same ssDNA conjugate concentrations as for HER2: TEVP-first DNA′, 20 nM; BLZ-second DNA′, 20 nM cTnI Ab1-first DNA, 5 nM; cTnI Ab2-second DNA, 5 nM.

FIG. 5 shows detection of thrombin and the specificity of PPR-based homologous assay. FIG. 5A is a schematic diagram illustrating a PPR-based homogeneous assay for thrombin detection. Aptamer1 binds to the fibrinogen-recognizing exosite of thrombin, and Aptamer2 binds to the heparin-binding exosite. FIG. 5B shows the relationship between absorbance signal and thrombin concentration. The inset graph shows the linear relationship at cTnI in the range from 0 to 1.25 nM. The assay was performed with 20 nM TEVP-first DNA′, 20 nM BLZ-second DNA′, 40 nM Aptamer1-first DNA, and 40 nM Aptamer2-second DNA. FIG. 2C shows the specificity of the three PPR-based homogeneous assays. Target proteins (HER2, cTnI or thrombin) at a concentration of 10 nM were analyzed under conditions optimized for each analysis method.

FIG. 6 shows detection of digoxigenin. FIG. 6A is a schematic diagram illustrating a PPR-based competitive homologous assay for detecting Digoxigenin (Dig). The proteolytic reaction with the anti-Dig antibody is inhibited by the presence of Dig and the degree of signal reduction is proportional to the Dig concentration. FIG. 2B shows the structures of digoxin and Dig. FIG. 2C shows the relationship between absorbance signal and Dig concentration. The inset graph shows the linear relationship between 1/ΔAbs and the Dig concentration ranging from 0 to 10.0 nM. The assay was performed with 10 nM TEVP-second DNA′, 10 nM BLZ-second DNA′, 2.5 nM Dig-second DNA, 2.5 nM Dig-second DNA, and 2.5 nM anti-Dig antibody.

FIG. 7 shows detection of antibodies. FIG. 7A is a schematic diagram illustrating a PPR-based immunoassay for detecting anti-Dig antibodies. FIG. 7B shows the relationship between absorbance signal and anti-Dig antibody concentration. The inset graph shows the linear relationship in the range from 0 to 10.0 nM of anti-Dig antibody. This assay was performed with 20 nM TEVP-first DNA′, 20 nM BLZ-second DNA′, 10 nM Dig-second DNA, and 10 nM Dig-second DNA. FIG. 7C shows the results of PPR-based immunoassay to detect anti-human chorionic gonadotropin (hCG) antibodies. The relationship between absorbance signal and anti-hCG concentration is shown. This assay was performed with 20 nM TEVP-second DNA′, 20 nM BLZ-second DNA′, 5 nM hCG-second DNA, and 5 nM hCG-second DNA.

FIG. 8 shows β-Lactamase zymogen (BLZ) and activation of β-Lactamase zymogen (BLZ) by tobacco etch virus protease (TEVP). BLZ was constructed by fusion of circularly substituted β-lactamase (BLA) through a linker cleavable by TEVP and the inhibitor protein thereof, β-lactamase inhibitory protein (BLIP). The BLIP protein was designed to have a very weak binding affinity for BLA and the cleavage reaction by TEVP prevented intramolecular interactions and activated BLZ. BLA has various chromogenic substrates, and CENTA™, which renders strong yellow (at 405 nm) through hydrolysis by BLA, was used in the examples of the present invention.

FIG. 9 shows the conjugation of BLZ with the second DNA′ (DNA2′). FIG. 9A shows the conjugation of 4-azido-l-phenylalanine (AzF)-incorporated β-lactamase zymogen (BLZ) with amine-modified second DNA′ through a bifunctional linker (N-hydroxysuccinimide ester-(polyethylene glycol)₄ -dibenzocyclooctyne; DBCO-PEG₄-NHS ester). DNA2′-amine was first reacted with the linker through reaction of amine and NHS ester, and then the resulting DNA2′-linker was conjugated with BLZ by a strain-promoted azide-alkyne reaction between the azide group of BLZ and the DBCO group of linker. FIG. 9B shows SDS-PAGE analysis of BLZ and BLZ-DNA2′.

FIG. 10 shows the production of TEV-DNA1′ (first DNA′) conjugates. FIG. 10A shows the first step, the conjugation of SpyCatcher, into which 4-azido-l-phenylalanine (AzF) is incorporated, with the amine-modified first DNA via the bifunctional linker (N-hydroxysuccinimide ester-(polyethylene glycol)₄-dibenzocyclooctyne; DBCO-PEG4-NHS ester). The DNA1′-amine was first reacted with the linker through the reaction between the amine and the NHS ester, and the resulting DNA1′-linker was conjugated to SpyCatcher by strain-promoted azide-alkyne reaction between the azide group of SpyCatcher and the DBCO group of the linker. FIG. 10B shows, as a second step, conjugation of SpyCatcher-DNA1′ and SpyTag-TEVP through a spontaneous isopeptide formation reaction between SpyCatcher and SpyTag. FIG. 10C shows SDS-PAGE analysis of SpyCatcher, SpyCatcher-DNA1′, SpyTag-TEVP and TEVP-DNA1′.

FIG. 11 shows the production of trastuzumab-DNA1 (first DNA) and pertuzumab-DNA2 (second DNA) conjugates. FIG. 11A shows conjugation of trastuzumab or pertuzumab with amine-modified ssDNA (DNA1-amine or DNA2-amine, respectively) via a bis-N-hydroxysuccinimide (NHS) ester linker. The amine-modified ssDNA was first reacted with an excess of the bis-NHS ester linker, and then the product containing the NHS ester group was conjugated with the amine group of the antibody. FIG. 11B shows SDS-PAGE analysis of trastuzumab, trastuzumab-DNA1, pertuzumab and pertuzumab-DNA2. FIG. 11C shows the ratio of ssDNA to antibody.

FIG. 12 shows optimization of reaction conditions. FIG. 12A shows the effects of MgCl₂ concentration in reaction buffer (137 mM NaCl, 2.7 mM KCl, 10 mM Na₂HPO₄, 2 mM KH₂PO₄, 10 mM DTT, and 0.5% (w/v) BSA; pH 7.0) on the difference in absorbance signal between 1 and 0 nM HER2 ectodomains. ΔΔAbs=ΔAbs (1 nM HER2)−ΔAbs (0 nM HER2). The absorbance signal (ΔAbs) is defined as the absorbance difference at 405 nm between 1 min and 60 min. FIG. 12B shows optimization of CENTA concentration in the reaction buffer. FIG. 12C shows concentration optimization of TEVP-DNA1′, BLZ-DNA2′, trastuzumab-DNA1 and pertuzumab-DNA2. For clarity, the amount of orange in color corresponds to the indicated value.

FIG. 13 shows the production of anti-cTnI antibody conjugates. FIG. 13A shows SDS-PAGE analysis of cTnI-Ab1, cTnI Ab1-DNA1, cTnI Ab2 and cTnI Ab2-DNA2. Two anti-cTnI antibodies (cTnI-Ab1 and cTnI-Ab2) were conjugated to DNA1 and DNA2, respectively, in the same manner as in conjugates of the anti-HER2 antibody and ssDNA. FIG. 13B shows the ratio of ssDNA to antibody.

FIG. 14 shows optimization of the concentrations of components of reaction to detect thrombin. FIG. 14A shows the conjugate structures of thrombin, Apatmer1 and Aptamer2. In addition, FIG. 14A shows the conjugate structure of thrombin and thrombin-binding aptamer (15-mer and 27-mer, Protein Data Bank [PDB] ID: 5ew2). FIG. 14B shows concentration optimization of TEVP-DNA1′, BLZ-DNA2′, Aptamer1-DNA1 and Aptamer2-DNA2. ΔΔAbs is defined as the difference in absorbance signal between 1 and 0 nM thrombin: ΔΔAbs=ΔAbs (1 nM thrombin)−ΔAbs (0 nM thrombin). The absorbance signal (ΔAbs) is defined as the absorbance difference at 405 nm between 1 min and 60 min.

FIG. 15 shows optimization of the concentrations of components of reaction for Dig detection. FIG. 15A shows the conjugation of digoxigenin-N-hydroxysuccinimide (Dig-NHS) ester with ssDNA-amine (DNA1 or DNA2). FIG. 15B shows optimization of digoxigenin-ssDNA conjugates and anti-digoxigenin concentrations. The concentration of TEVP-DNA1′ and BLZ-DNA2′ is 10 nM. ΔΔAbs=ΔAbs(1 nM digoxigenin)−ΔAbs (0 nM digoxigenin). FIG. 15C shows a time-dependent absorbance response to various concentrations of digoxigenin.

FIG. 16 shows optimization of concentrations of components of reaction for anti-Dig antibody detection. FIG. 16 shows optimization of the concentrations of digoxigenin-ssDNA conjugates and enzyme-ssDNA conjugates. ΔΔAbs=ΔAbs (2 nM anti-Dig antibody)−ΔAbs (0 nM anti-Dig antibody).

FIG. 17 shows the production of hCG and ssDNA conjugates (hCG-DNA1 and hCG-DNA2). FIG. 17A shows anti-hCG antibody detection. FIG. 17B shows conjugation of hCG with amine-modified ssDNA (DNA1-amine or DNA2-amine) via a bis-N-hydroxysuccinimide (NHS) ester linker. The amine-modified ssDNA was first reacted with an excess of the bis-NHS ester linker, and then the product having the NHS ester group was conjugated to the amine group of hCG (PDB ID: 1 hcn). FIG. 17C shows SDS-PAGE analysis of hCG, hCG-DNA1 and hCG-DNA2. FIG. 17D shows the ratio of ssDNA to hCG. FIG. 17E shows optimization of concentrations of hCG-ssDNA conjugates and enzyme-ssDNA conjugates. ΔΔAbs=ΔAbs (2 nM anti-hCG antibody)−ΔAbs (0 nM anti-hCG antibody).

### DETAILED DESCRIPTION OF THE INVENTION AND PREFERRED EMBODIMENTS

Unless defined otherwise, all technical and scientific terms used herein have the same meanings as appreciated by those skilled in the field to which the present invention pertains. In general, the nomenclature used herein is well-known in the art and is ordinarily used.

In the present invention, a first DNA and a second DNA were linked to a first binder and a second binder binding to a target substance, respectively, a first DNA′ and a second DNA′ having sequences complementary to the first DNA and the second DNA were linked to a protease and a zymogen, respectively, and then the target substance was detected using a composition containing the four ssDNA-conjugates and a substrate specific to the enzyme source (zymogen) (Example 1).

Accordingly, in one aspect, the present invention is directed to a composition for detecting a target substance containing i) a first DNA-first binder conjugate in which a first DNA is linked to a first binder; ii) a first DNA′-protease conjugate in which a first DNA′ having a sequence complementary to the first DNA is linked to a protease; iii) a second DNA-second binder conjugate in which a second DNA is linked to a second binder; iv) a second DNA′-zymogen conjugate in which a second DNA′ having a sequence complementary to the second DNA is linked to an enzyme source (zymogen); and v) a substrate specific to the enzyme source.

In the present invention, the first binder and the second binder may be the same as or different from each other.

In the present invention, the binder may be an antibody, an aptamer, an antigen, a small molecule, or a protein that is capable of binding to the target substance.

In the present invention, the antibody may be trastuzumab, pertuzumab or anti-cTnI antibody, and the antigen may be digoxigenin (Dig) or human chorionic gonadotropin (hCG).

In the present invention, the target substance may be an antigen, antibody, small molecule or protein.

In the present invention, the antigen may be HER2, cTnI or thrombin, and the antibody may be an anti-digoxigenin (Dig) antibody or an anti-human chorionic gonadotropin (hCG) antibody.

In the present invention, when the target substance is HER2, the first binder is trastuzumab and the second binder is pertuzumab (Examples 2 and 3).

In the present invention, when the target substance is cardiac troponin I (cTnI), the first binder is a first anti-cTnI antibody and the second binder is a second anti-cTnI antibody (Example 4).

In the present invention, when the target substance is thrombin, the first binder is a first aptamer capable of binding to thrombin and the second binder is a second aptamer capable of binding to thrombin (Example 5).

In the present invention, the first aptamer is represented by SEQ ID NO: 7, the second aptamer is represented by SEQ ID NO: 8, the first DNA is represented by SEQ ID NO: 5, and the second DNA is represented by SEQ ID NO: 6 (Table 2).

In the present invention, when the target substance is an anti-digoxigenin (Dig) antibody, the first binder and the second binder are digoxigenin (Dig) (Example 7).

In the present invention, when the target substance is an anti-human chorionic gonadotropin (hCG) antibody, the first binder and the second binder are human chorionic gonadotropin (hCG) (Example 7).

The affinity of the binder for the target substance and the ratio of TEVP or BLZ to the binder may affect assay performance. The dissociation constant (Ka) of the binders to HER2 and thrombin have been reported (trastuzumab to HER2: 173 pM; pertuzumab to HER2: 32 pM; Aptamer1 to thrombin: 102.6 nM; Aptamer2 to thrombin: 0.5 nM) (Deng, B. et al. Anal Chim Acta 837, 1-15, do i:10.1016/j.aca.2014.04.055 (2014); Komarova, T. V. et al. Sci Rep 9, 16168, doi:10.1038/s41598-019-52507-9 (2019)), the number of ssDNA molecules covalently linked to the binder is different, 1 (thrombin aptamer) to 4 (trastuzumab and pertuzumab). In order to offset the difference, a method for detecting HER2 and thrombin was developed by optimizing the concentrations of the four components (TEVP-first DNA′, BLZ-second DNA′, first binder-first DNA, and second binder-second DNA). Under each selected condition, both assays showed similar performance in detection range and LOD. In addition, other binders (anti-cTnI antibody, anti-Dig antibody, and polyclonal anti-hCG antibody) having no reported affinity may be applied to PPR-based homogeneous analysis, by controlling the concentration of the components thereof. These results suggest that many available binders with Kd values in the nanomolar range are useful for PPR-based homogeneous assays. In addition, since PPR-based homogeneous assays are performed in a module manner, it is easy for those skilled in the art to apply the homogeneous assay of the present invention to other types of target substances.

The binder to the target substance was linked to TEVP and BLZ through hybridization between the two DNA strands, instead of direct conjugation. By conjugating binders with ssDNA through this approach, two types of binders can be used: proteins (including antibodies) and aptamers. In addition, since there is no need to prepare the TEVP-first binder and the BLZ-second binder prior to mixing with the sample, the assay may be performed in a one-step manner. Since DNA oligomers may be synthesized to contain specific functional groups for chemical reactions, various pathways for generating DNA and conjugates have been reported (Dovgan, I., et al., Bioconjug Chem 30, 2483-2501, doi:10.1021/acs.bioconjchem.9b00306 (2019)). Further defined ssDNA-binder conjugates can be produced using other chemical reactions, in addition to NHS-amine coupling, which contribute to the robustness and reproducibility of the assay.

Detecting molecular interactions such as protein-protein interactions may be performed using binders that target different molecules. In addition, since binders such as antibodies and aptamers have been reported to recognize post-translational modifications of proteins (Diaz-Fernandez, A. et al. Chemical Science 11, 9402-9413, doi:10.1039/d0sc00209g (2020)), such modifications can be detected in the target protein. In addition, using a reported binder for modified nucleic acids (Bhattacharjee, R., et al., Analyst 143, 4802-4818, doi: 10.1039/c8an01348a (2018)), epigenetic modifications of chromosomes can be detected at specific positions.

In the present invention, when the target substance is a small molecule, the first binder and the second binder may be the same small molecule as the target substance and the composition further contains an anti-small molecule antibody.

In the present invention, when the small molecule is digoxigenin (Dig), the anti-small molecule antibody may be an anti-Dig antibody (Example 6).

In the present invention, the first DNA may be represented by SEQ ID NO: 1 and the second DNA may be represented by SEQ ID NO: 2 (Table 1).

In the present invention, the protease may be a tobacco etch virus (TEV) protease, a hepatitis C virus (HCV) protease, a tobacco vein mottling virus (TVMV) protease or a human rhinovirus (HRV) 3c protease, but is not limited thereto.

In the present invention, the enzyme source may have a configuration in which an enzyme is linked to an enzyme activity inhibitor protein through a peptide linker that is cleavable by the protease.

In the present invention, the enzyme source may be β-lactamase zymogen.

In the present invention, the substrate may be a

chromogenic substrate and the chromogenic substrate may be CENTA™, but is not limited thereto.

In another aspect, the present invention is directed to a method of detecting a target substance including (a) mixing the composition with a sample containing a target substance, (b) binding the target substance to the first binder of the first DNA-first binder conjugate, binding the target substance to the second binder of the second DNA-second binder conjugate, hybridizing the first DNA linked to the first binder with the first DNA′ linked to a protease, and hybridizing the second DNA linked to the second binder with the second DNA′ linked to an enzyme source (zymogen), and (c) detecting a signal generated by a proximity proteolysis reaction between the first DNA′-protease conjugate hybridized with the first DNA of the first binder and the second DNA′-zymogen conjugate hybridized with the second DNA of the second binder.

Homogeneous analytical procedures for proteins and small molecules may be performed in a mix-and-read format at a constant temperature based on the PPR-based analytical principle. In addition, the catalytic conversion of CENTA™ by β-lactamase enables the detection of target substances at sub-nanomolar concentrations based on absorbance signals.

In the present invention, the enzyme source may have a configuration in which the enzyme is linked to the enzyme activity inhibitor protein through a peptide linker that is cleavable by the protease.

In the present invention, the proximal proteolysis reaction in step (d) is performed by cleaving the peptide linker by the protease to split the enzyme source into an enzyme and an enzyme activity inhibitor protein to thereby activate the enzyme, and hydrolyzing the substrate by the activated enzyme to generate a signal.

In the present invention, the protease may be a tobacco etch virus (TEV) protease, a hepatitis C virus (HCV) protease, a tobacco vein mottling virus (TVMV) protease, or a human rhinovirus (HRV) 3c protease, but is not limited thereto.

In the present invention, the enzyme source may be β-lactamase zymogen.

In the present invention, the substrate may be a chromogenic substrate and the chromogenic substrate may be CENTA™, but is not limited thereto.

In the present invention, the enzyme source is β-lactamase zymogen, the substrate is CENTA™and the change in absorbance at 405 nm when the sample contains the target substance is greater than the change in absorbance when the sample does not contain the target substance.

In the present invention, the enzyme source is β-lactamase zymogen, the substrate is nitrocefin, and the sample generates a red signal when it contains the target substance and generates a yellow signal when it does not contain the target substance.

In another aspect, the present invention is directed to a method of detecting a small molecule including (a) mixing the composition further containing the anti-small molecule antibody with a sample containing a target substance, (b) hybridizing the first DNA linked to the first binder with the first DNA′ linked to a protease and hybridizing the second DNA linked to the second binder with the second DNA′ linked to an enzyme source (zymogen), and (c) detecting whether or not the small molecule is present depending on the presence or absence of a signal generated by a proximity proteolysis reaction between the hybridized first DNA′-protease conjugate and the hybridized second DNA′-zymogen conjugate.

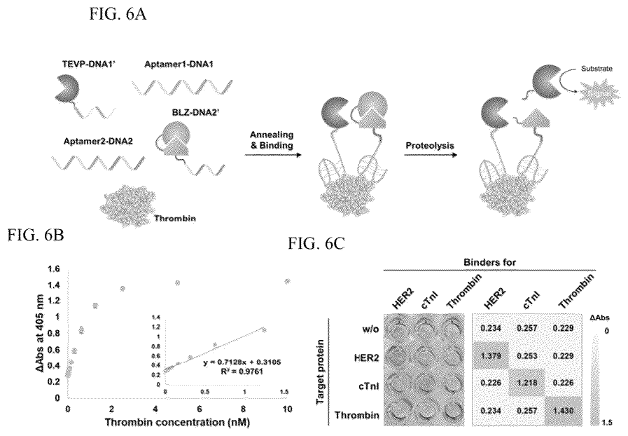

In the present invention, when the sample in step (a) contains a small molecule, the small molecule binds to an anti-small molecule antibody, so that the proximity proteolysis reaction in step (c) does not occur and there is no signal generated by the reaction (Example 6, FIG. 6).

In the present invention, when the sample in step (a) does not contain a small molecule, the anti-small molecule antibody binds to the first binder of the first DNA-first binder conjugate and to the second binder of the second DNA-second binder conjugate, so that a signal is generated by the proximity proteolysis reaction between the hybridized first DNA′-protease conjugate and the hybridized second DNA′-zymogen conjugate in step (c) (Example 6, FIG. 6).

Descriptions of components related to the first binder, the second binder, the target substance, the first DNA, and the second DNA used in the method for detecting the target substance or small molecule according to the present invention may be equally applied to the method.

Hereinafter, the present invention will be described in more detail with reference to examples. However, it will be obvious to those skilled in the art that these examples are provided only for illustration of the present invention, and should not be construed as limiting the scope of the present invention.

### EXAMPLE

### Example 1: Design of PPR-Based Homogeneous Assays

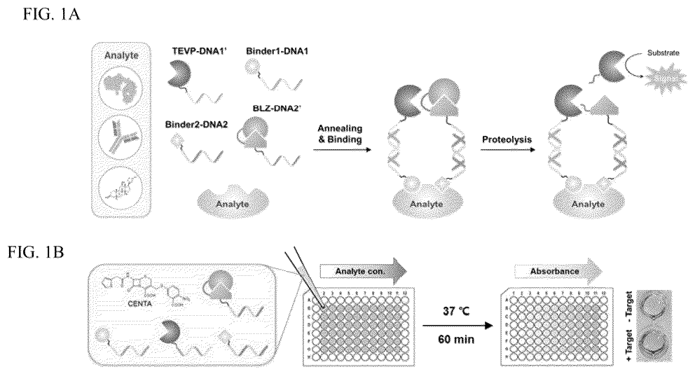

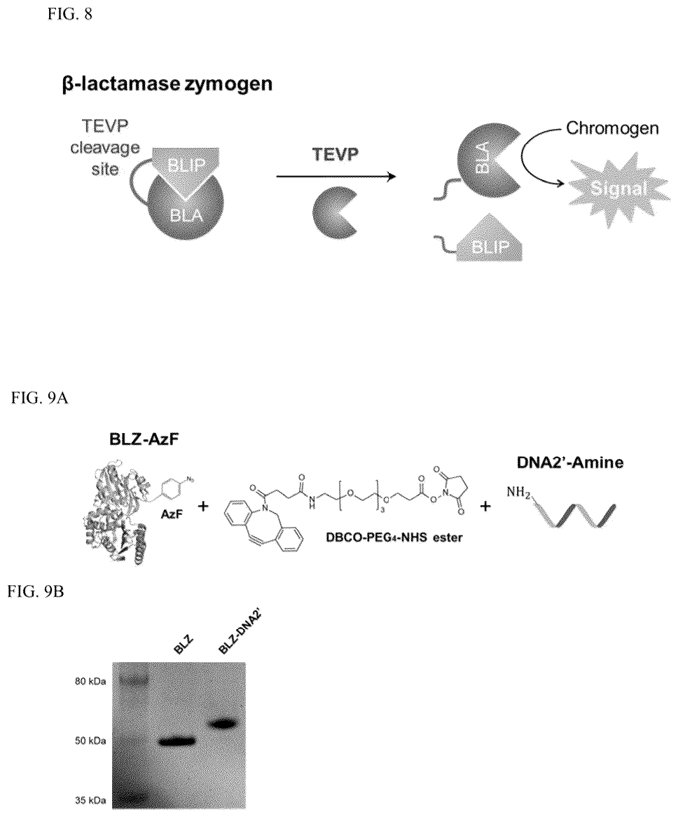

Proteases and zymogens are physically linked to binders such as antibodies, aptamers, and antigens for analysis using specific and robust hybridization between complementary ssDNA molecules. A pair of a tobacco etch virus protease (TEVP) and β-lactamase zymogen (BLZ) were used for proteolysis. TEVP has been widely used in recombinant protein engineering due to high specificity thereof. BLZ was previously designed to be activated by TEVP (FIG. 8). Both proteins are also orthogonal to the human system, which is essential for actual sample applications. Four conjugates (TEVP-first DNA′, BLZ-second DNA′, first binder-first DNA and second binder-second DNA) and a chromogenic substrate for β-lactamase (CENTA™ ) were mixed with the sample and changes in absorbance were monitored. The general assay was performed in a one-step format and the signal (absorbance at 405 nm for hydrolyzed CENTA™ ) was measured after 1 hour (FIG. 1B).

In a homogeneous assay to detect the target substance, samples were mixed with four conjugates (TEVP-first DNA′, BLZ-second DNA′, first binder-first DNA agent, second binder-second DNA) and 400 mM CENTA™ (EMD Millipore, USA) in a reaction buffer (137 mM NaCl, 2.7 mM KCl, 10 mM Na₂HPO₄, 2 mM KH₂PO₄, 40 mM MgCl₂, 10 mM DTT, 0.5% (w/v) BSA, and pH 7.4). Hydrolysis of CENTA™ by β-lactamase was monitored by measuring absorbance (A₄₀₅) at 405 nm using a plate reader (synergy HT multi-detection reader; BioTek Instruments, USA) at 37° C. for 1 hour. The absorbance signal (ΔAbs=A₄₀₅ at 60 min−A₄₀₅ at 1 min) was plotted as a function of the concentration of the target substance. All experiments were performed at least three times and the limit of detection (LOD) was calculated as the sum of the mean ΔAbs of the blank and three times the standard deviation. In the competitive immunoassay for small molecule detection, an antibody against the target was further contained in the reaction solution. The following proteins were used as targets: HER2 (extracellular domain; 10004-H₀₈H; Sino Biological, China), cTnI protein (ab207624; Abcam, United Kingdom), thrombin (ab62452; Abcam), digoxigenin (D9026; Sigma, USA), digoxigenin polyclonal antibody (PA1-85378; Thermo Fisher Scientific, USA) and hCG monoclonal antibody (MA5-14680; Thermo Fisher Scientific).

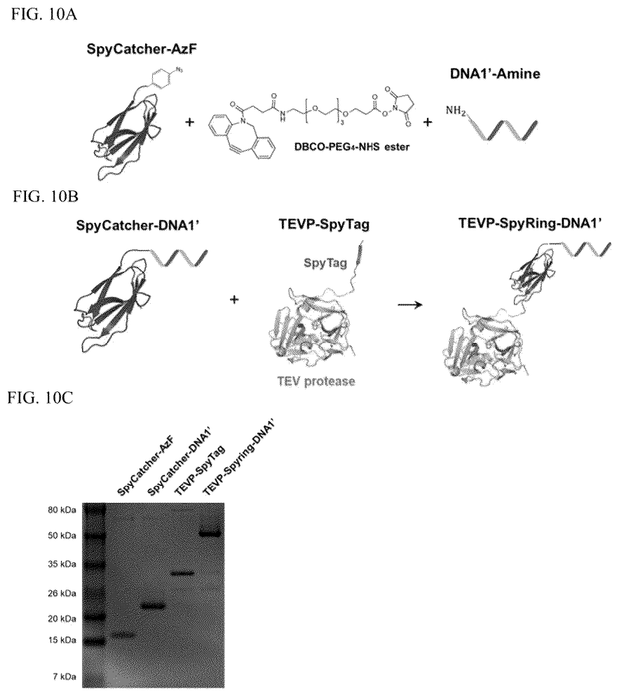

TEVP and BLZ were linked to binders using nucleic acid hybridization rather than direct conjugation in order to develop a method applicable to various binders. For site-specific conjugation between BLZ and the second DNA′ (Table 1), a bifunctional linker (N-hydroxysuccinimide ester-(polyethyleneglycol)4-dibenzocyclooctyne; DBCO-PEG4-NHS ester) was used (FIG. 9). The β-lactamase zymogen was engineered to contain 4-azido-L-phenylalanine (AzF) and the synthesized second DNA′ was functionalized using an amine group. The SpyTag/Catcher system was used to conjugate the first DNA′ with TEVP and it was previously observed that the BLZ-second DNA′ binding method resulted in significant inactivation of the protease. SpyCatcher containing AzF was conjugated with the first DNA′ using the DBCO-PEG4-NHS ester (Table 1) and formation of a spontaneous isopeptide bond between SpyTag and SpyCatcher generated TEVP-first DNA′ (FIG. 10).

The production of binder-ssDNA molecules differs depending on the binder and methods for each case will be described later in the following Example. The sequence of the ssDNA molecule was designed to provide strong and specific interactions between the strands under assay conditions and was analyzed using the nucleic acid package.

TABLE 1
Amine-modified DNAs

| | | SEQ ID |
|---|---|---|
| ssDNA | Sequence (5′→3′) | NO: |
| First DNA | [Amine] CCTAACTTGACGATACAGCG | 1 |
| Second DNA | [Amine] TGGAATGCTGGTGGCGTAGG | 2 |
| First DNA′ | [Amine] CGCTGTATCGTCAAGTTA | 3 |
| Second DNA′ | [Amine] CCTACGCCACCAGCATTC | 4 |

### Example 2: Homologous Assay to Detect Ectodomain of HER2

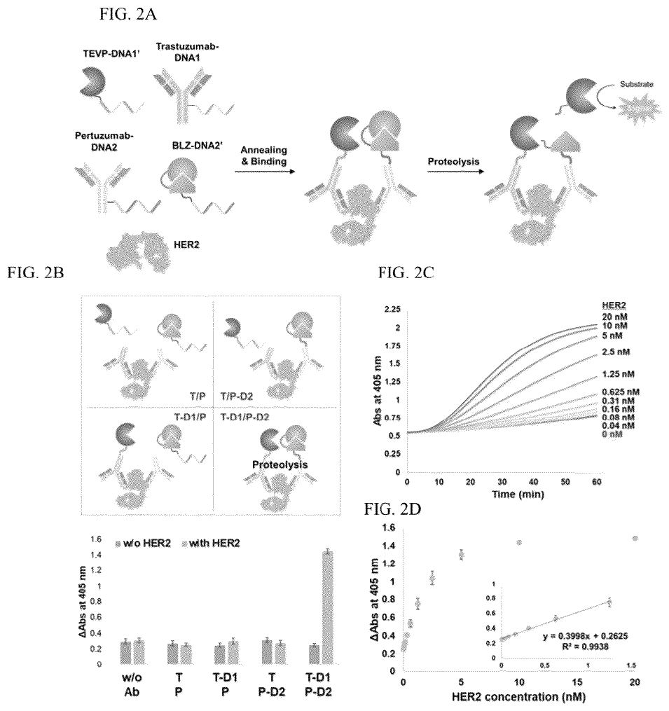

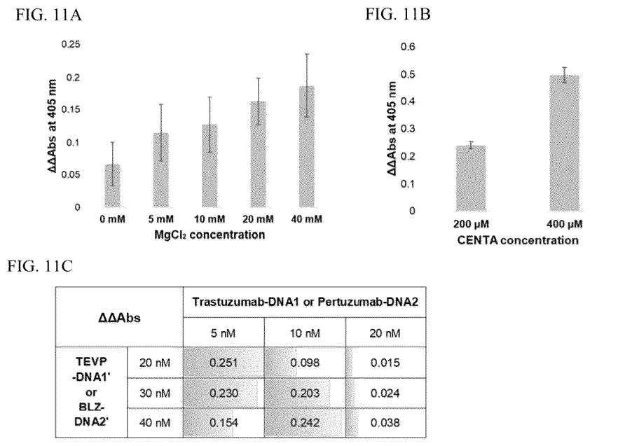

A homologous assay based on PPR was performed to detect the ectodomain of human epidermal growth factor receptor-2 (HER2) (FIG. 2A), which has been reported to be overexpressed in some cancer cells. Two approved monoclonal antibodies, namely, trastuzumab and pertuzumab, which bind to extracellular domain IV and domain II, respectively, were selected as binders to HER2. The antibodies expressed using HEK293F cells were covalently modified with ssDNA (first DNA or second DNA, Table 1) to form trastuzumab-first DNA and pertuzumab-second DNA (FIG. 11). Antibody-ssDNA conjugates were produced using the reaction between amine and N-hydroxysuccinimide (NHS) ester (FIG. 11A); the amine-modified ssDNA first reacted with an excess of the Bis-NHS ester linker and then the product having NHS ester was conjugated with an antibody. Since an antibody molecule has one or more amine groups, the approach inevitably produced heterogeneous products (FIGS. 11B and 11C). However, antibody-ssDNA conjugates were produced using this method to utilize a variety of commercially available antibodies. In addition, an assay was performed to detect cardiac troponin I using two antibodies from the manufacturer.

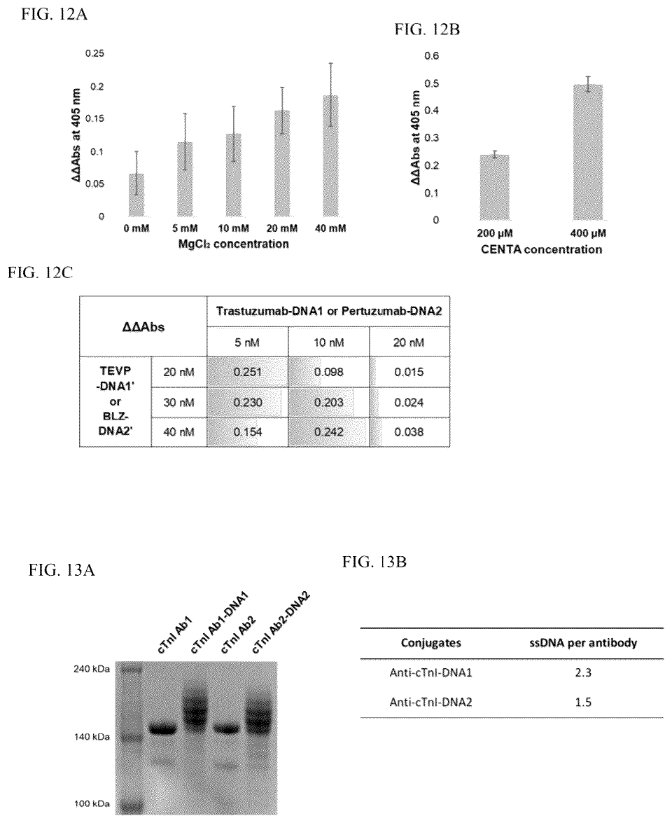

Whether or not an assay generated a specific signal for the ectodomain of HER2 was determined using the antibody-ssDNA conjugate (FIG. 2B). Various antibody combinations were tested as TEV-first DNA′ and BLZ-second DNA′ pairs. The result showed that conjugation of each ssDNA with an anti-HER2 antibody is necessary and sufficient to detect the HER2 protein. The absorbance signal (ΔAbs) is defined as the difference between absorbance values at 1 min and 60 min at 405 nm. In addition, the concentrations of reaction components including CENTA™, antibody-ssDNA conjugate, TEV-first DNA′, BLA-second DNA′ and MgCl₂ were optimized (FIG. 12). HER2 ectodomain concentrations in samples were quantified under optimized conditions (FIG. 2C). The absorbance at 405 nm generated by hydrolysis of CENTA™ was monitored for 1 hour and a signal difference of 20 nM or less for HER2 was observed (FIGS. 2C and 2D). The linear relationship between absorbance difference and HER2 concentration was observed in the range of 0 to 1.25 nM and the limit of detection (LOD) was 5.03 pM (FIG. 2E).

### Example 3: Detection of HER2 in Cell Membranes

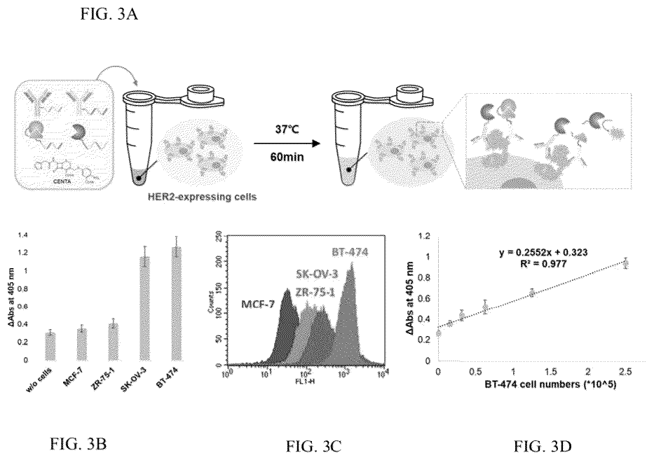

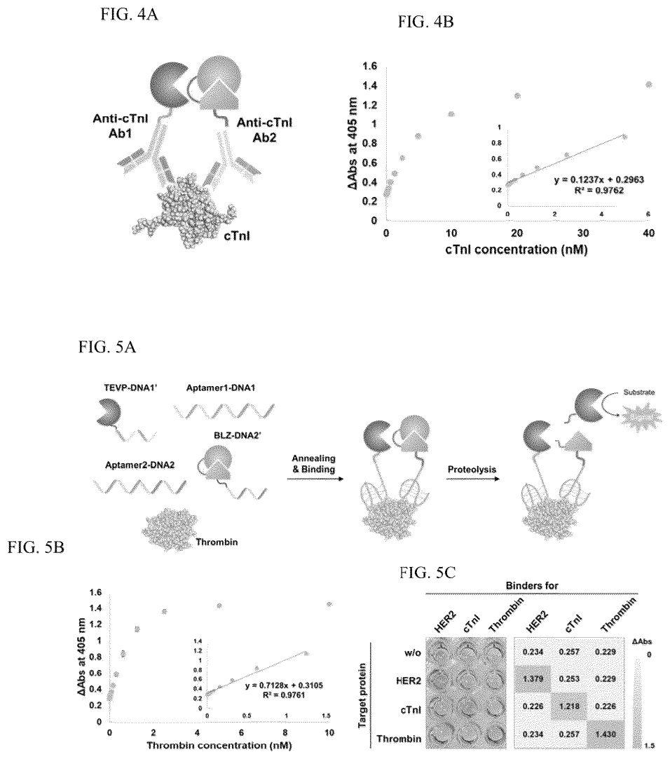

HER2 is a membrane protein and the expression thereof is usually analyzed using flow cytometry or immunohistochemical staining including a plurality of steps including immobilization and repeated washing. In the present invention, a one-step analysis procedure for detecting HER2 was performed in several breast cancer cell lines having various expression levels of HER2 (FIG. 3A). As in the case of analyzing the soluble ectodomain of HER2, four conjugates (TEVP-first DNA′, BLZ-second DNA′, trastuzumab-first DNA, pertuzumab-second DNA) and CENTA™ were added to a solution containing suspended cells, and the absorbance signal was measured 1 hour later. The signal increased in the order of MCF-7, ZR-75-1, SK-OV-3, and BT-474 (FIG. 3B), which corresponded to the results of flow cytometry (FIG. 3C). In addition, the linear relationship between the signal and the number of cells was observed in the range of 0-2.5×10⁵ cells using the BT-474 cell, and the LOD was 5.75×10³ cells (FIG. 3D). Samples were prepared by treating cells with trypsin and the method described is useful for analysis of membrane proteins in substantially intact cells.

### Example 4: Homogeneous Assay to Detect cTnI

A number of antibodies against various antigens have already been constructed or developed and homogeneous assays based on PPR using the antibodies as binders have been developed. Cardiac troponin I (cTnI) in the blood acts as an essential biomarker for heart damage and protein detection methods for detecting the same have been researched. In the present invention, PPR-based homogeneous assays for detecting cTnI using two commercially available anti-cTnI antibodies that recognize distinct epitopes corresponding to amino acids 23-29 (anti-cTnI Ab1) and 41-49 (anti-cTnI Ab2) of cTnI were developed. In accordance with the procedure used for the anti-HER2 antibody, the antibody was conjugated with ssDNA (first DNA and second DNA) to form anti-cTnI Ab1-first DNA and anti-cTnI Ab2-second DNA (FIG. 13). The results of analysis using the antibody-ssDNA conjugate showed a linear relationship with the concentration-dependent curve in the range of 0 to 5.0 nM cTnI and the LOD was 10.51 pM (FIG. 2E). Thus, it can be seen that other antibodies available for various antigens can be readily used to develop PPR-based homogeneous assays.

### Example 5: Homogeneous Assay Using Aptamer to Detect Thrombin and Specificity of Homogeneous Assay

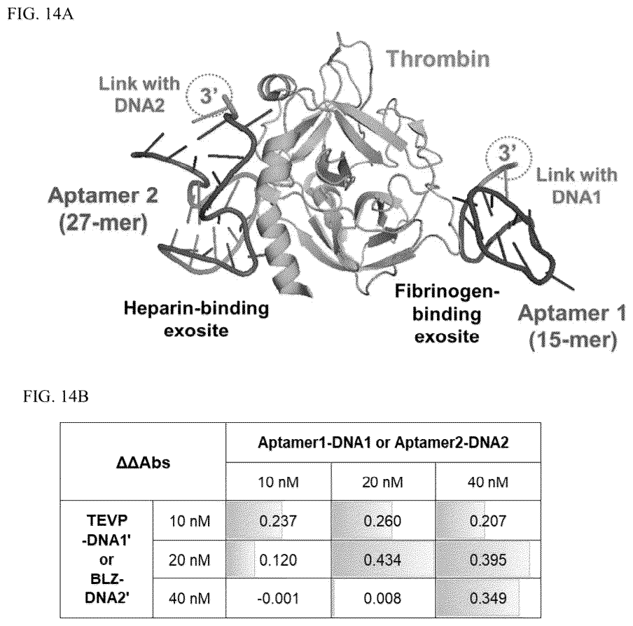

Aptamers consisting of nucleic acids have been developed to have affinity for various molecules. Compared to general antibodies, aptamers have advantages such as small size, high stability, and production through chemical synthesis. In the present invention, the ssDNA-binder could be synthesized without additional conjugation and purification steps using an aptamer as a binder in the analysis. Two aptamers reported to bind to separate regions of human α-thrombin (15-mer and 27-mer DNA) were used. The 15-mer thrombin aptamer (aptamer 1) interacts with the fibrinogen-recognition exosite, whereas the 27-mer aptamer (aptamer 2) binds to the heparin-binding exosite (FIG. 14A). A PPR-based homologous assay for detecting thrombin was performed using the synthesized aptamer1-first DNA and aptamer2-second DNA (Table 2) (FIG. 5A). Optimization was required for the concentrations of ssDNA-reporter and ssDNA-binder because the ratio of reporter to binder was different from that of the assay using an antibody as the binder (FIG. 14B). A hyperbolic curve was obtained by plotting the difference in absorbance at 405 nm as a function of thrombin concentration, a linear relationship was observed up to 1.25 nM, and the LOD was 6.82 pM (FIG. 5B). The results show that aptamers can be used as binders for PPR-based homologous assays.

The specificity of three PPR-based homogeneous assays developed to detect HER2, cTnI and thrombin was evaluated. Signals differed significantly from the background only when the binders matched the targets thereof (FIG. 5C). Thus, it can be seen that a specific homogeneous assay can be developed using a binder suitable for the target substance.

TABLE 2
Thrombin aptamer DNA

| | | SEQ ID |
|---|---|---|
| Type | Sequence (5′→3′) | NO: |
| First DNA | TAACTTGACGATACAGCG | 5 |
| Second DNA | GAATGCTGGTGGCGTAGG | 6 |
| First aptamer | GGTTGGTGTGGTTGG | 7 |
| Second aptamer | AGTCCGTGGTAGGGCAGGTTGG | 8 |
| | GGTGAC | |
| First aptamer- | GGTTGGTGTGGTTGGttttttT | 9 |
| first DNA* | AACTTGACGATACAGCG | |
| Second | AGTCCGTGGTAGGGCAGGTTGG | 10 |
| aptamer-second | GGTGACttttttGAATGCTGGT | |
| DNA* | GGCGTAGG | |

*Underlined sequences represent thrombin aptamers, and lowercase letters represent spacers between the aptamer and first DNA or second DNA.

### Example 6: Competitive Homologous Assay to Detect Digoxigenin

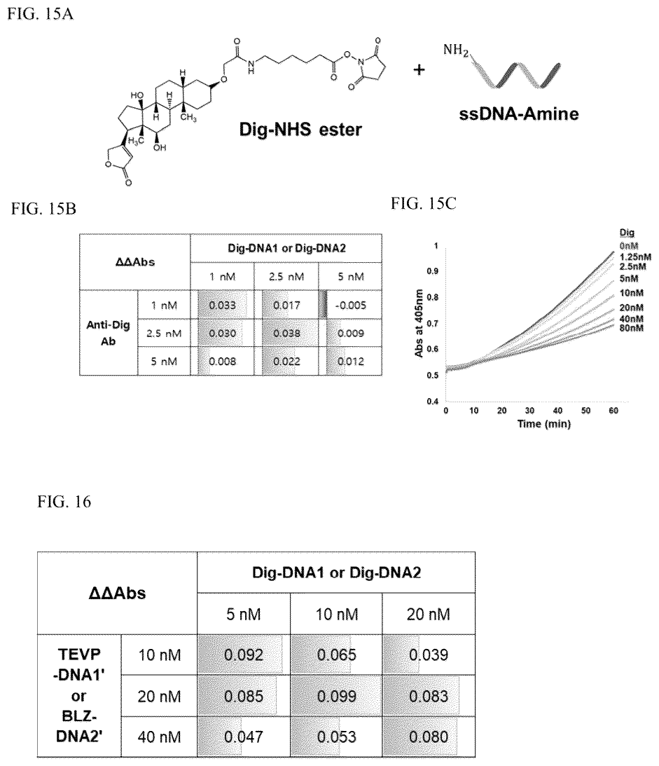

Unlike biological macromolecules such as proteins, with a few exceptions, finding pairs of binders that simultaneously interact with small molecules is generally limited (H Ueda, K. T., et al., Nat Biotechnol 14, 1714-1718, doi:10.1038/nbt1296-1714. (1996)). Therefore, competition-based assays such as competitive ELISA have been developed to detect low molecular weight targets. A competitive homologous assay to detect small molecules using a PPR-based assay format was developed (FIG. 6A). The chemical substance, digoxin has been used to treat a variety of heart diseases and serum levels thereof should be monitored due to potential toxicity caused by overdose. Digoxigenin (Dig) is a part of digoxin (FIG. 6B) and was used to develop the method of the present invention. The first DNA and the second DNA were conjugated with digoxigenin NHS-ester (FIG. 15A) for association with TEVP-first DNA′ and BLZ-second DNA′, respectively. The proteolysis reaction between TEVP-Dig and BLZ-Dig is enhanced by binding to an anti-Dig antibody, but inhibited by digoxin in the sample. The concentrations of TEVP-first DNA′, BLZ-second DNA′, Dig-first DNA, Dig-second DNA and anti-Dig antibody were optimized (FIG. 15B). The absorbance signal at 405 nm decreased with increasing Dig concentration (FIG. 15C), and a linear relationship between 1/ΔAbs and Dig concentration was observed in the range of 0-10 nM Dig, and the LOD was 273.9 pM (FIG. 6C).

### Example 7: Homogeneous Assay to Detect Antibodies

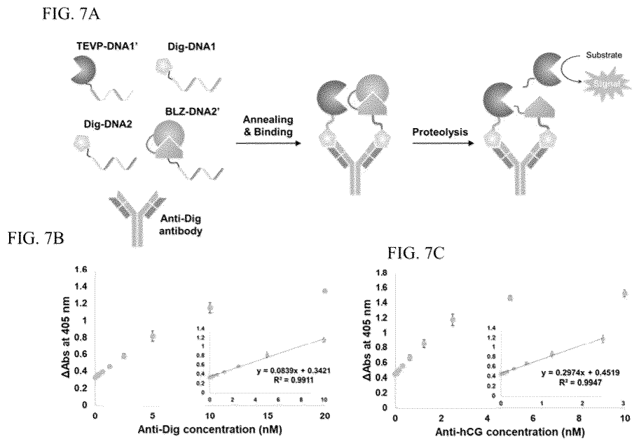

The concentrations of antibodies against target antigens provide essential information for understanding the clinical response of therapeutic agents and diagnosing infectious and autoimmune diseases. In addition, the antibody titer of the medium is a key parameter controlling the production process of therapeutic antibodies. Reagents (Dig-first DNA and Dig-second DNA) generated for the competitive homologous assay for Digoxin were used to detect anti-Dig antibodies in the samples (FIG. 7A). The concentrations of TEVP-first DNA′, BLZ-second DNA′, Dig-first DNA, and Dig-second DNA were optimized (FIG. 16). The difference in absorbance at 405 nm increased as the anti-Dig antibody concentration increased to 20 nM, and a linear curve was obtained in the range of 0-10 nM and the LOD was 78.51 pM (FIG. 7B).

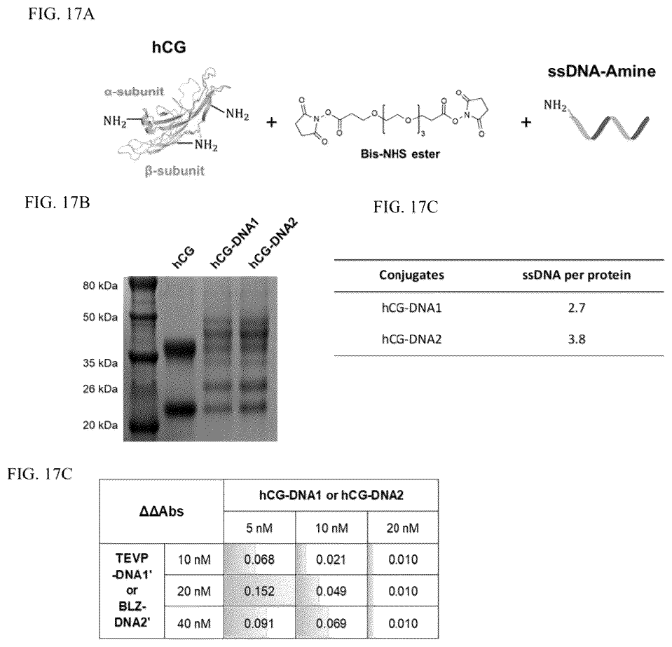

The size of the antigen may affect the proteolytic reaction between TVEP and BLZ combined with the antibody, and in the present invention, the method of the present invention was applied to an antibody against a protein antigen (human chorionic gonadotropin, hCG, FIG. 17A). hCG was conjugated to either the first DNA or the second DNA using an NHS-amine coupling reaction (FIGS. 17B-17D), and the reagent concentrations were also optimized (FIG. 17E). Concentration-response curves for the anti-hCG antibody were obtained in the range of 0 to 10 nM and the LOD was 9.83 pM (FIG. 7C). These results showed that the homogeneous assay platform based on PPR of the present invention can be applied to detect antibodies against various antigens including autoantigens ranging from small molecules to large proteins.

### Example 8: Test Method

### Example 8-1: Expression and Purification of Protein

### Tobacco Etch Virus Protease-SpyTag (TEVP-SpyTag)

E. coli BL21 (DE3) was transfected with TEVP-SpyTag, a plasmid (pSPEL515) encoding a fusion protein composed of TEVP and SpyTag. Cultures grown in 2×YT medium at 37° C. were induced with 0.4 mM isopropyl β-d-1-thiogalactopyranoside (IPTG) at an optical density (OD₆₀₀) of 0.5 and then further grown at 25° C. for 8 hours. The cells were collected by centrifugation and proteins (containing His6 tag) were purified using Ni-NTA resin (Clontech, Japan) in accordance with the manufacturer's instructions. Purified TEVP-SpyTag protein was stored in TEVP storage buffer (50 mM Tris-HCl, 10 mM NaCl, 0.5 mM EDTA, and 40% (v/v) glycerol; pH 8.0) at −20° C.

E. coli BL21 (DE3) was transfected with plasmids expressing SpyCatcher-AzF and the b-lactamase zymogen-azidophenylalanine (BLZ-AzF) SpyCatcher-AzF (pSPEL517) or BLZ-AzF (pSPEL427) along with two other plasmids: first, an orthogonal pair of aminoacyl-tRNA synthetase and tRNA from Methanococcus jannaschii were encoded to incorporate AzF in response to the TAG codon and second, E. coli prolyl-tRNA synthetase (ProRS) was overexpressed to inhibit misincorporation of AzF into the Pro position of the protein. Cells were grown to an OD₆₀₀ of 0.5 at 37° C. in 2×YT and 0.2% L-arabinose and 50 nM anhydrous tetracycline were added thereto to induce expression of orthogonal pairs and ProRS. When the OD₆₀₀ reached 1.0, 1 mM AzF and 0.4 mM IPTG were added to the culture to induce expression of SpyCatcher-AzF or BLZ-AzF. Cells expressing SpyCatcher-AzF were further incubated at 30° C. for 8 hours and cells expressing BLZ-AzF were incubated at 25° C. overnight. The protein has a His₆ tag and was purified on Ni-NTA resin in accordance with the manufacturer's instructions. BLZ-AzF was expressed in the periplasmic space and the purification procedure was applied to the periplasmic fraction. Purified proteins were stored at −20° C. in PBS containing 20% (v/v) glycerol.

IgG (Trastuzumab and Pertuzumab) and Human Chorionic Gonadotropin (hCG)

For the construction of expression vectors, synthetic genes encoding trastuzumab, pertuzumab and hCG including Kozak sequences were cloned into pcDNA 3.1 at NotI and XhoI sites. The names of the constructed plasmid including each plasmid and protein sequences are shown in Table 3. Proteins were produced in the HEK293F cell line maintained in FreeStyle 293 expression medium (Gibco, Thermo Fisher Scientific, USA). For protein expression, 2×10⁶ cells were transfected with 250 μg of the expression vector encoding the protein sequence using 750 μg of polyethyleneimine (Polysciences, USA) in 200 mL of medium. The cells were incubated for 5 to 7 days, the supernatant was collected using centrifugation, each antibody was purified on CaptivA protein A affinity resin (Repligen, USA), and hCG was purified on NI-NTA resin in accordance with the instructions provided by the manufacturer. Each purified protein was stored at −20° C. in PBS.

TABLE 3
Protein sequence

| | | SEQ |
|---|---|---|
| | | ID |
| Type | Sequence (5′→3′) | NO: |
| TEVP- | MGSSHHHHHHGSWSHPQFEKKLGGGSGGGSAHIVMVDAYKPT | 11 |
| SpyTag | KGGGSGGGSEFESLFKGPRDYNPISSTICHLTNESDGHTTSLYGI | |
| (pSPEL515) | GFGPFIITNKHLFRRNNGTLVVQSLHGVFKVKNTTTLQQHLIDGR | |
| | DMIIIRMPKDFPPFPQKLKFREPQREERICLVTTNFQTKSMSSMV | |
| | SDTSCTFPSGDGIFWKHWIQTKDGQCGSPLVSTRDGFIVGIHSAS | |
| | NFTNTNNYFTSVPKNFMELLTNQEAQQWVSGWRLNADSVLWGGHK | |
| | VFMVKPEEPFQPVKEATQLMN | |
| SpyCatc | MGSSHHHHHHGSGGGSXKLGGGSGGGSAMVDTLSGLSSEQGQ | 12 |
| her-AzF* | SGDMTIEEDSATHIKESKRDEDGKELAGATMELRDSSGKTISTWI | |
| (pSPEL517) | SDGQVKDFYLYPGKYTFVETAAPDGYEVATAITFTVNEQGQVTVN | |
| | GKATKGDAHI | |
| BLZ-AzF* | MKYLLPTAAAGLLLLAAQPAMAMGXGGSGGSAGVMTGAKFTQ | 13 |
| (pSPEL468) | IQFGMTRQQVLDIAGAENCETGGSFGDSIHCRGHAAGDYYAYATF | |
| | GFTSAAADAKVDSKSQEKLLAPSAPTLTLAKFNQVTVGMTRAQVL | |
| | ATVGQGSCTTWSEYYPAYPSTAGVTLSLSCFDVDGYSSTGAYRGS | |
| | AHLWFTDGVLQGKRQWDLVGSGGGSGGGSENLYFQGGGGSGGGSK | |
| | LMDERNRQIAEIGASLIKHWGGGGGHPETLVKVKDAEDQLGARVG | |
| | YIELDLNSGKILESFRPEERFPMMSTFKVLLCGAVLSRIDAGQEQ | |
| | LGRRIHYSQNDLVEYSPVTEKHLTDGMTVRELCSAAITMSDNTAA | |
| | NLLLTTIGGPKELTAFLHNMGDHVTRLDRWEPELNEAIPNDERDT | |
| | TTPVAMATTLRKLLTGELLTLASRQQLIDWMEADKVAGPLLRSAL | |
| | PAGWFIADKSGAGERGSRGIIAALGPDGEPSRIVVIYTTGSQALE | |
| | HHHHHH | |
| Trastuzumab, | MGWSCIILFLVATATGVHSEVQLVESGGGLVQPGGSLRLSCA | 14 |
| heavy chain | ASGFNIKDTYIHWVRQAPGKGLEWVARIYPTNGYTRYADSVKGRF | |
| (pSPEL874) | TISADTSKNTAYLQMNSLRAEDTAVYYCSRWGGDGFYAMDYWGQG | |
| | TLVTVSSASTKGPSVFPLAPSSKSTSGGTAALGCLVKDYFPEPVT | |
| | VSWNSGALTSGVHTFPAVLQSSGLYSLSSVVTVPSSSLGTQTYIC | |
| | NVNHKPSNTKVDKKVEPKSCDKTHTCPPCPAPELLGGPSVELFPP | |
| | KPKDTLMISRTPEVTCVVVDVSHEDPEVKENWYVDGVEVHNAKTK | |
| | PREEQYNSTYRVVSVLTVLHQDWLNGKEYKCKVSNKALPAPIEKT | |
| | ISKAKGQPREPQVYTLPPSRDELTKNQVSLTCLVKGFYPSDIAVE | |
| | WESNGQPENNYKTTPPVLDSDGSFFLYSKLTVDKSRWQQGNVFSC | |
| | SVMHEALHNHYTQKSLSLSPGK | |
| Trastuzumab, | MGWSCIILFLVATATGVHSDIQMTQSPSSLSASVGDRVTITC | 15 |
| light chain | RASQDVNTAVAWYQQKPGKAPKLLIYSASFLYSGVPSRESGSRSG | |
| (pSPEL875) | TDFTLTISSLQPEDFATYYCQQHYTTPPTFGQGTKVEIKRTVAAP | |
| | SVFIFPPSDEQLKSGTASVVCLLNNFYPREAKVQWKVDNALQSGN | |
| | SQESVTEQDSKDSTYSLSSTLTLSKADYEKHKVYACEVTHQGLSS | |
| | PVTKSENRGEC | |
| Pertuzumab, | MGWSCIILFLVATATGVHSEVQLVESGGGLVQPGGSLRLSCA | 16 |
| heavy chain | ASGFTFTDYTMDWVRQAPGKGLEWVADVNPNSGGSIYNQRFKGRF | |
| (pSPEL624) | TLSVDRSKNTLYLQMNSLRAEDTAVYYCARNLGPSFYFDYWGQGT | |
| | LVTVSSASTKGPSVFPLAPSSKSTSGGTAALGCLVKDYFPEPVTV | |
| | SWNSGALTSGVHTFPAVLQSSGLYSLSSVVTVPSSSLGTQTYICN | |
| | VNHKPSNTKVDKKVEPKSCDKTHTCPPCPAPELLGGPSVELFPPK | |
| | PKDTLMISRTPEVTCVVVDVSHEDPEVKENWYVDGVEVHNAKTKP | |
| | REEQYNSTYRVVSVLTVLHQDWLNGKEYKCKVSNKALPAPIEKTI | |
| | SKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCS | |
| | VMHEALHNHYTQKSLSLSPG | |
| Pertuzumab, | MGWSCIILFLVATATGVHSDIQMTQSPSSLSASVGDRVTITC | 17 |
| light chain | KASQDVSIGVAWYQQKPGKAPKLLIYSASYRYTGVPSRESGSGSG | |
| (pSPEL625) | TDFTLTISSLQPEDFATYYCQQYYIYPYTFGQGTKVEIKRTVAAP | |
| | SVFIFPPSDEQLKSGTASVVCLLNNFYPREAKVQWKVDNALQSGN | |
| | SQESVTEQDSKDSTYSLSSTLTLSKADYEKHKVYACEVTHQGLSS | |
| | PVTKSFNRGEC | |
| hCG, | MDYYRKYAAIFLVTLSVFLHVLHSAPDVQDCPECTLQENPLF | 18 |
| α-subunit | SQPGAPILQCMGCCFSRAYPTPLRSKKTMLVQKNVTSESTCCVAK | |
| (pSPEL706) | SYNRVTVMGGFKVENHTACHCSTCYYHKSHHHHHH | |
| hCG, | MEMFQGLLLLLLLSMGGTWASKEPLRPRCRPINATLAVEKEG | 19 |
| β-subunit | CPVCITVNTTICAGYCPTMTRVLQGVLPALPQVVCNYRDVRFESI | |
| (pSPEL707) | RLPGCPRGVNPVVSYAVALSCQCALCRRSTTDCGGPKDHPLTCDD | |
| | PRFQDSSSSKAPPPSLPSPSRLPGPSDTPILPQGSHHHHHH | |

*X represents the position of the protein into which AzF is incorporated.

### Determination of Protein Concentration

The concentration of the purified protein was determined by measuring the absorbance at 280 nm and the extinction coefficient was calculated through the ProtParam site (http://web.expasy.org/protparam/).

### Example 8-2: Preparation of Single-Stranded DNA (ssDNA) Conjugates

Conjugation of ssDNA and protein through a Bifunctional Linker (N-hydroxysuccinimide ester-(polyethylene glycol)4-dibenzocyclooctyne; DBCO-PEG4-NHS ester)

Single-stranded DNA functionalized with 5′ amine groups (DNA1′ or DNA2′) was mixed with a 20-fold molar excess of the DBCO-PEG4-NHS ester. The reaction mixture of PBS was incubated at 25° C. in the dark for 2 hours. The modified ssDNA was purified by 75% ethanol precipitation to remove unreacted linkers and the pellet was resuspended in PBS for storage at −20° C. 5′-modified DNA2′ (DNA2′-DBCO) and BLZ-AzF were mixed at a molar ratio of 5:1 for a strain-promoted azide-alkyne reaction between DBCO and azide. The reaction mixture in PBS was incubated at 4° C. overnight. The product was first purified by anion exchange chromatography on a HiTrap Q column (GE Healthcare Life Sciences, USA) to remove unconjugated protein, and the conjugate was eluted with a gradient of 0.2 to 1.0 M NaCl. Then, the purified fraction was subjected to gel filtration chromatography on Superdex 75 10/300 GL (GE Healthcare Life Sciences, USA) to remove unreacted DNA2′-DBCO. Purified BLZ-DNA2 conjugates were stored at −20° C. in PBS containing 20% (v/v) glycerol. Conjugation of SpyCatcher-AzF with DNA1′-DBCO was performed in the same manner as in BLZ-DNA2′ and the product was purified using a HiTrap Q column with gradient elution from 0.2 to 1.0 M NaCl. The purified solution contains unreacted DNA1′-DBCO that does not interfere with the subsequent reaction between SpyCatcher and SpyTag. Finally, partially purified SpyCatcher-DNA1′ was incubated along with TEVP-SpyTag for spontaneous isopeptide bond formation. Unreacted SpyCatcher-DNA1′ was removed using a Step-Tactin resin (IBA Lifesciences, Germany) in accordance with the manufacturer's instructions. The TEVP-SpyTag protein has a Strep tag. Purified BLZ-DNA2 conjugates were stored at −20° C. in TEVP storage buffer.

Conjugation of ssDNA and Protein via bis-N-hydroxysuccinimide (NHS) Linker

The amine-functionalized ssDNA (DNA1 or DNA2) synthesized in Bioneer Co. (Korea) was mixed with a 200-fold molar excess of the Bis-NHS ester linker and the reaction mixture was incubated in PBS at 4° C. for 1 hour. The modified ssDNA was precipitated with 75% ethanol to remove unreacted linkers and the pellet was resuspended in PBS. ssDNA having an NHS ester group was reacted with a trastuzumab, pertuzumab, anti-cardiac troponin I (cTnI) antibody (ab92408, ab10231; Abcam, United Kingdom) or hCG at 4° C. for 16 hours. The ratio of ssDNA to protein for the conjugation reaction was 25:1 for the trastuzumab, 45:1 for the pertuzumab, 55:1 for the anti-cTnI antibody, and 30:1 for the hCG. The conjugate was purified by gel filtration chromatography on Superdex 200 Increase 10/300 GL (GE Healthcare Life Sciences, USA) to remove unreacted ssDNA. Purified conjugates were stored at -20° C. in PBS.

Conjugation of ssDNA and ε-(digoxigenin-3-0-acetamido)caproic acid N-hydroxysuccinimide Ester (Dig-NHS Ester)

Amine-functionalized ssDNA (DNA1 or DNA2) was incubated along with a 20-fold molar excess of Dig-NHS ester (Sigma, USA) in PBS at 25° C. for 2 hours. Dig-modified ssDNA was precipitated with 75% ethanol to remove unreacted Dig-NHS esters. The pellet was resuspended in PBS and the solution was stored at −20° C. The conjugate concentration was calculated from the absorbance measured at 260 nm and the extinction coefficient calculated on the MOLBIOTOOLS site (http://www.molbiotools.com/dnacalculator.html).

Determination of ssDNA-Protein Conjugate Concentration

The concentration of each conjugate was calculated

by measuring absorbance at 260 and 280 nm as follows (using extinction coefficients calculated from MOLBIOTOOLS and ProtParam sites).

A_{260,conjugate}=A_{260,DNA}+A_{260,Protein}=ε_{260,DNA}×b×c_{260,DNA}+ε_{260,Protein}×b×c_{260,Protein}

A_{280,conjugate}=A_{280,DNA}+A_{280,Protein}=ε_{280,DNA}×b×c_{280,DNA}+ε_{280,Protein}×b×c_{280,Protein}
wherein ε: extinction coefficient (M⁻¹ cm⁻¹), b: path length (cm), c: concentration (M).

### Example 8-3: Detection of HER2 in the Plasma Membrane

Human breast cancer cell lines BT-474, SK-OV-3, ZR-75-1 and MCF-7 were maintained at 37° C. in a humidified atmosphere containing 5% CO₂ in RPMI 1640 (HyClone, USA) supplemented with 10% fetal bovine serum (FBS) and 1% penicillin/streptomycin solution. To detect HER2 using the assay, after treatment with trypsin, the cells were washed with PBS and resuspended in reaction buffer containing 80 pM Dynasore (dynamin inhibitor; ab120629; Abcam, United Kingdom) to adjust the concentration to 0.5×10⁶ cells/mL. HER2 detection was performed using a PPR-based homologous assay.

### Example 8-4: Flow-Cytometric Analysis

After treatment with trypsin, the cells were washed twice with 1 mL cold fluorescence-activated cell sorting (FACS) buffer (PBS, 2% FBS) and incubated along with 100 nM trastuzumab on ice for 1 hour. Then, the cells were washed twice with cold FACS buffer and incubated on ice along with 10 ng/mL of a goat anti-human IgG (H+L) cross-adsorbed secondary antibody conjugated with Alexa Fluor 488 (A-11013; Invitrogen, Thermo Fisher, USA) for 1 hour. After washing, the cells were resuspended in 500 μL of cold FACS buffer and the cell surface fluorescence intensity was analyzed by FACS (BD FACSCalibur, BD Biosciences, USA).

### INDUSTRIAL APPLICABILITY

The method for detecting a target substance according

to the present invention is fast and simple because it is performed by a one-step process including simultaneously adding four conjugates (a first DNA-first binder conjugate, a first DNA′-protease conjugate, a second DNA-second binder conjugate, and a second DNA′-zymogen conjugate) and a substrate specific to the enzyme source to a sample using proximity proteolysis. In addition, the detection is possible and high sensitivity is obtained even when the concentration of the target substance is less than one nanomole. In addition, the binders are linked via two ssDNAs, rather than directly binding to the protease and the enzyme source, thus eliminating the necessity to prepare the protease-first binder and enzyme source-second binder conjugates before mixing with the sample. Therefore, the present invention can be used in a variety of fields such as disease diagnosis and drug concentration monitoring and is highly versatile by repeatedly using the same ssDNA-protease conjugate and ssDNA-enzyme source conjugate, and detecting various biomarkers only by changing the binder. Also, the present invention can be used to detect post-translational modifications of proteins or epigenetic modifications of chromosomes at specific locations.

Although specific configurations of the present invention have been described in detail, those skilled in the art will appreciate that this description is provided to set forth preferred embodiments for illustrative purposes, and should not be construed as limiting the scope of the present invention. Therefore, the substantial scope of the present invention is defined by the accompanying claims and equivalents thereto.

### SEQUENCE LISTING FREE TEXT

An electronic file is attached.

1. A method for detecting a target substance treating composition comprising:
i) a first DNA-first binder conjugate in which a first DNA is linked to a first binder;
ii) a first DNA′-protease conjugate in which a first DNA′ having a sequence complementary to the first DNA is linked to a protease;
iii) a second DNA-second binder conjugate in which a second DNA is linked to a second binder;
iv) a second DNA′-zymogen conjugate in which a second DNA′ having a sequence complementary to the second DNA is linked to an enzyme source (zymogen); and
v) a substrate specific to the enzyme source.

2. The method according to claim 1, wherein the first binder and the second binder are the same as or different from each other.

3. The method according to claim 1, wherein the first binder and the second binder are each independently an antibody, an aptamer, an antigen, a small molecule, or a protein that is capable of binding to the target substance.

4. The method according to claim 3, wherein the antibody is a trastuzumab, pertuzumab or anti-cTnI antibody; and
the antigen is digoxigenin (Dig) or human chorionic gonadotropin (hCG).

5. The method according to claim 1, wherein the target substance is an antigen, an antibody, a small molecule or a protein.

6. The method according to claim 5, wherein the antigen is HER2, cTnI or thrombin; and
the antibody is an anti-digoxigenin (Dig) antibody or an anti-human chorionic gonadotropin (hCG) antibody.

7. The method according to claim 1, wherein when the target substance is HER2, the first binder is trastuzumab and the second binder is pertuzumab.

8. The method according to claim 1, wherein when the target substance is cardiac troponin I (cTnI), the first binder is a first anti-cTnI antibody and the second binder is a second anti-cTnI antibody.

9. The method according to claim 1, wherein when the target substance is thrombin, the first binder is a first aptamer capable of binding to thrombin and the second binder is a second aptamer capable of binding to thrombin.

10. The method according to claim 9, wherein the first aptamer is represented by SEQ ID NO: 7, the second aptamer is represented by SEQ ID NO: 8, the first DNA is represented by SEQ ID NO: 5, and the second DNA is represented by SEQ ID NO: 6.

11. The method according to claim 1, wherein when the target substance is an anti-digoxigenin (Dig) antibody, the first binder and the second binder are digoxigenin (Dig).

12. The method according to claim 1, wherein when the target substance is an anti-human chorionic gonadotropin (hCG) antibody, the first binder and the second binder are human chorionic gonadotropin (hCG).

13. The method according to claim 1, wherein when the target substance is a small molecule, the first binder and the second binder are the same small molecule as the target substance and the composition further comprises an anti-small molecule antibody.

14. The method according to claim 13, wherein when the small molecule is digoxigenin (Dig), the anti-small molecule antibody is an anti-Dig antibody.

15. The method according to claim 1, wherein the first DNA is represented by SEQ ID NO: 1, and the second DNA is represented by SEQ ID NO: 2.

16. The method according to claim 1, wherein the protease is a tobacco etch virus (TEV) protease, a hepatitis C virus (HCV) protease, a tobacco vein mottling virus (TVMV) protease, or a human rhinovirus (HRV) 3c protease.

17. The method according to claim 1, wherein the enzyme source has a configuration in which an enzyme is linked to an enzyme activity inhibitor protein through a peptide linker that is cleavable by the protease.

18. The method according to claim 1, comprising:
(a) mixing the composition with a sample containing a target substance;
(b) binding the target substance to the first binder of the first DNA-first binder conjugate, binding the target substance to the second binder of the second DNA-second binder conjugate, hybridizing the first DNA linked to the first binder with the first DNA′ linked to a protease, and hybridizing the second DNA linked to the second binder with the second DNA′ linked to an enzyme source (zymogen); and
(c) detecting a signal generated by a proximity proteolysis reaction between the first DNA′-protease conjugate hybridized with the first DNA of the first binder and the second DNA′-zymogen conjugate hybridized with the second DNA of the second binder.

19. (canceled)

20. The method according to claim 18, wherein the proximal proteolysis reaction of step (c) comprises:
activating the enzyme by separating the enzyme source into an enzyme and an enzyme activity inhibitor protein by cleavage of the peptide linker by the protease; and
generating a signal by the activated enzyme hydrolyzing a substrate.

21. (canceled)

22. The method according to claim 18, wherein the enzyme source is β-lactamase zymogen, the substrate is CENTA™, and a change in absorbance at 405 nm when the sample contains the target substance is greater than a change in absorbance when the sample does not contain the target substance.

23. The method according to claim 18, wherein the enzyme source is β-lactamase zymogen, the substrate is nitrocefin, and the sample generates a red signal when the sample contains the target substance and generates a yellow signal when the sample does not contain the target substance.

24. The method according to claim 13, comprising:
(a) mixing the composition with a sample containing a small molecule;
(b) hybridizing the first DNA linked to the first binder with the first DNA′ linked to a protease, and hybridizing the second DNA linked to the second binder with the second DNA′ linked to an enzyme source (zymogen); and
(c) detecting whether or not a small molecule is present depending on presence or absence of a signal generated by a proximity proteolysis reaction between the hybridized first DNA′-protease conjugate and the hybridized second DNA′-zymogen conjugate.

25. The method according to claim 24, wherein when the sample in step (a) contains a small molecule, the small molecule binds to an anti-small molecule antibody, so that the proximity proteolysis reaction in step (c) does not occur and there is no signal generated by the reaction.

26. The method according to claim 24, wherein when the sample in step (a) does not contains a small molecule, the anti-small molecule antibody binds to the first binder of the first DNA-first binder conjugate and the second binder of the second DNA-second binder conjugate, so that the proximity proteolysis reaction between the hybridized first DNA′-protease conjugate and the hybridized second DNA′-zymogen conjugate in step (c) generate a signal.
